(19) United States
(12) Patent Application Publication
(10) Pub. No.: US 2021/0315942 A1
(43) Pub. Date: Oct. 14, 2021

(54) Dr. Lord's Anti-Covid-19 Formula & Energy Elixir

(71) Applicant: Roxanne Simone Lord, Jamaica, NY (US)

(72) Inventor: Roxanne Simone Lord, Jamaica, NY (US)

(21) Appl. No.: 16/845,004

(22) Filed: Apr. 9, 2020

(51) Int. Cl. A61K 35/644; A61K 36/288; A61K 36/63; A61K 36/28; A61K 36/80; A61K 36/185; A61K 36/9068; A61K 36/16; A61K 36/79

(52) U.S. Cl. CPC A61K 35/644; A61K 36/288; A61K 36/63; A61K 36/28; A61K 36/79; A61K 36/185; A61K 36/9068; A61K 36/16; A61K 36/80

(57) ABSTRACT

The invention consists of two formulas, Dr. Lord's Olive Leaf Tea Formula (Anti-Covid-19 Tea Formula) and the Energy Elixir. Both formulas are unique combinations of plants that have been proven to combat and provide relief from viruses, flu symptoms, itchy and sore throat. My invention is specially formulated to work fast, provide fast relief, in a matter of 20-30 minutes in most cases. One should use promptly when feeling the beginning, the onset of these symptoms. My invention, these formulas, work well together to facilitate speedy system recovery and functionality. The olive leaf tea formula is in loose tea form and the Energy Elixir is in liquid form.

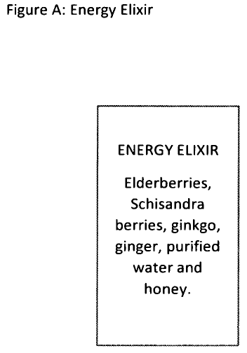

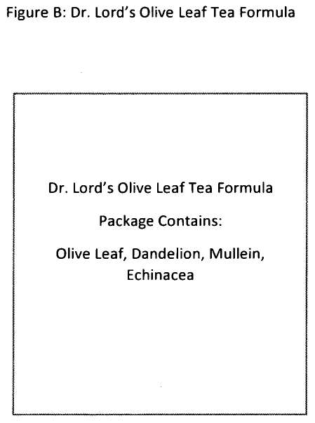

### BACKGROUND OF THE INVENTION

I made this formula (a) during the coronavirus pandemic of 2020 to protect myself and my family. I am a doctor of naturopathy, a graduate of Trinity School of Natural Medicine. I am also a doctor of ministry and humanity. I have studied plant medicine and have studied the bible. The book of Revelation made mention of a tree whose leaves were for the healing of the nations. I made the connection that these leaves are for such a time as this. (Covid-19 Apr. 9, 2020)

I made invention b, the Energy Elixir for a businessman who got sick. I needed to boost his immune system, and feed minerals into his body as we managed the stress he was under at work.

Dr. Lord's Olive Leaf Tea Formula (a) helps the body fight viruses and speeds up recovery time. It supports vitality and greater health.

Energy Elixir Formula (b) works well with Dr. Lord's Olive Leaf Tea Formula (a) to build up or strengthen the body and mind.

### BRIEF SUMMARY OF INVENTION

Invention (a) Dr. Lord's Olive Leaf Tea Formula is an herbal combination of herbs, olive leaf, dandelion, mullein and echinacea that when steeped into tea helps the body to fight viruses, parasites and bacteria. Supports general immune boost and detoxification, fast eradication of bugs, virus & mucus.

(B) Dr. Lord's Energy Elixir is a combination of elderberries, with Schisandra berries, ginger, ginkgo, honey with purified water. Boosts the brain, boosts immune function, and manage stress hormones.

Figure A: Brief Description: The Energy Elixir is a dark red liquid packaged in a clear glass bottle that has a label and is sealed. The formula consists of elderberry, ginger, ginkgo, Schisandra berries, honey and purified water.

Figure B: Brief Description: Dr. Lord's Olive Leaf Tea Formula containing mullein, dandelion, echinacea and olive leaf herbs. It is packed in a zip locked bag for freshness.

### DETAILED DESCRIPTION OF THE INVENTION:

Dr. Lord's Olive Leaf Tea Formula is a tea blend of four herbs that are known for thousands of years for their anti-viral and immune boosting properties. Olive Leaf comes from the olive tree that gives olive oil and olives. The leaves are anti-viral and also knocks out flu symptoms in about 20 minutes after drinking it. It is blended in with other herbs that support immune function such as Echinacea, dandelion supports detoxification or cleaning of the blood. Mullein also helps to knock out flu symptoms such as runny nose and itchy or sore throat. The invention A is a tea and can be sweetened with honey for taste. We have packaged the Olive Leaf Tea Formula in a 4 ounce zip locked bag for freshness and convenience. Each serving is 1 teaspoon to 8 ounces of water.

Invention B, works well the Olive Leaf Tea Formula and is called the Energy Elixir. It has been bottled and sealed for freshness and preservation of the ingredients. The ingredients are elderberries, schisandra berries, ginger, ginkgo, honey and purified water. These ingredients are known for immune support, managing stress hormones, and boosting brain support.

1. What I claim as my invention is a tea/herbal formula, called Dr. Lord's Olive Leaf Tea Formula that supports optimum health by helping the body fight off flu and viruses, as well as shortens recovery time from flu and viruses.
What I claim as my invention is that my special combination of berries and herbs, Formula B, the Energy Elixir, supports the immune system, helps the body manage stress, and acts as a general tonic to support optimum health.
